(19) United States
(12) Patent Application Publication
(10) Pub. No.: US 2004/0056201 A1
(43) Pub. Date: Mar. 25, 2004

(54) Food surface sanitation hood

(76) Inventors: Ronald G. Fink, Jupiter, FL (US); Walter Ellis, Jupiter, FL (US); Charles Pearsall, Stuart, FL (US)

Correspondence Address: MALIN HALEY AND DIMAGGIO, PA, 1936 S ANDREWS AVENUE, FORT LAUDERDALE, FL, 33316, US

(21) Appl. No.: 10/065,131

(22) Filed: Sep. 19, 2002

(51) Int. Cl. G01J 5/02

(52) U.S. Cl. 250/352

(57) ABSTRACT

This invention is a modular, adjustable, easy to maintain, portable food sanitation hood system, comprising a hooded means for subjecting food to sanitizers including UV light, ozone and hydroxyl radicals, and a method for using the system. The means for subjecting food to the sanitizers includes one or more UV radiation sources and one or more target rods for UV radiation located under a hood. The UV radiation sources are preferably low-vapor mercury UV light sources that emit UV light of approximately 185 to 254 nm. The hood preferably includes an adjustable light curtain to at least partially reduce radiation emitted away from the food. The target rods comprise up to approximately up to 0-30% titanium dioxide, up to 0-30% silver and up to 0-30% copper, by weight. The system may include a mister for adding mist in proximity to the food for efficient sanitization.

### BACKGROUND OF INVENTION

1. Field of the Invention

The present invention relates to sanitation of a food surface and more particularly pertains to industrial, modular, compact, and efficient systems and devices for sanitization of foods during processing, especially those processed on assembly lines. The present invention also relates to foods sanitized by such systems and devices.

2. Description of Related Art

Food sanitation is a growing concern in the world. More and more aggressive disease-causing agents are discovered every year. Moreover, an increasing number of people are made ill each year by eating contaminated foods. Also, the numbers of foods linked to food-caused illnesses continues to increase. Nonetheless, the desire for safer foods is higher than ever. In fact, more and more state and federal jurisdictions are requiring that businesses use the most efficacious food safety and sanitation practices.

Efficient use of modern sanitization techniques decreases the cost of applying them. These savings, when passed to the consumer, mean the consumers spend less on the processed foods. Also, efficient sanitizing techniques can allow more food to be processed in a smaller amount of space. Thus smaller facilities may compete with larger ones, thereby increasing competition between processors and lowering prices to consumers. In addition, facilities that use less-safe processing techniques may be easily retrofit to install a modular and efficient apparatus. Also, the modularity of a new sanitizing system or device makes it easy to manufacture and easy to replace parts.

Sanitizing radiation allows a highly controllable application of organism-killing radiation to foods and food additives. The use of sanitizing radiation in the food industry in general is well known in the prior art, and has been used in a variety of forms, including gamma ray radiation, ultraviolet (UV) light and infrared radiation.

For example, it is well known that the use of gamma radiation and UV radiation has been used in some countries for the sterilization of spices and animal feeds. However, over-use of ultraviolet radiation may cause undesirable chemical reactions with a food or food additive, which can cause the food or food additive to obtain undesirable flavors or textures. Also, various vitamins and proteins may be altered or destroyed through being subjected to too much radiation, reducing the food value of the treated product.

Traditional methods of irradiating foods processed in assembly line manner and other prepackaged foods use an unnecessary amount of space in a food processing facility. Furthermore, they fail to take full advantage of the combined use of ozone, UV light and hydroxyl radicals in the sanitation process. For example, U.S. Pat. No. 6,150,663 to Rosenthal teaches a system incorporating an irradiation system using radiation, and only radiation, to sanitize food. Rosenthal teaches vibration of the food to optimize exposure of the food to the radiation. The entire system is inefficient and ineffective, as irradiation alone may not properly sanitize food. For example, if there were a slight fold on the food surface, the shaded food surface would not be sanitized at all. However, ozone and hydroxyl radical ions are able to get beneath a fold and sanitize the folded over food surface. Moreover, if the food were prepackaged so that vibration would topple the packaging, then the Rosenthal process would be contraindicated.

The lack of a modular system makes utilization of the sanitizing combination of UV light, ozone and hydroxyl radicals difficult to apply or manufacture economically, or to retrofit into an existing system. There is no system designed with modularity in mind, to suit different processes properly, or to provide easy manufacture or replacement of component parts. There is no system wherein the radiation, ozone and hydroxyl radicals are optimized for food treatment. There is no system that efficiently utilizes sanitizing radiation, ozone and hydroxyl radicals to sanitize processed and/or prepackaged foods. There is no system which adds moisture to the sanitizing system to improve the efficiency of the formation of hydroxyl radicals in sanitizing food. There is no system that is easy to assemble, adjust, transport, clean, maintain and disassemble. There is no system using sanitizing radiation, ozone and hydroxyl radicals that is adjustable on a frame, or that has an adjustable light curtain so high levels of the radiation may be used in a small amount of space and still be safe for operators. There is no system that takes advantage of the sanitizing and free-moving characteristics of ozone and hydroxyl radicals to improve sanitization beyond what mere radiation can perform.

### SUMMARY OF INVENTION

The present invention is a modular, adjustable, portable, easy to maintain food sanitation hood system, comprising a hooded means for subjecting food to sanitizers including UV light, ozone and hydroxyl radicals, having one or more UV light sources and one or more target rods located under the hooded means in optical proximity to the UV light sources. The UV radiation source emits UV light of approximately 185 to 254 nm, and is a low-vapor or high-vapor mercury UV light sources that emit UV light of approximately 185 to 254 nm. In another embodiment, the hooded means has drainage holes through a top surface. The target rods are approximately 0-30% titanium dioxide, up to 0-30% silver and up to 0-30% copper, by weight. In yet another embodiment, the system has a mister for adding mist in proximity to the target rods for the efficient production of hydroxyl radicals. In still another embodiment, hydroxyl radicals are generated in part from the moisture in the ambient air in the proximity of the target rods.

In another embodiment, the system of claim 1 has at least one mounting tab located on the outer surface of one side of the hooded means, and a connector tab connecting at least two mounting tabs on one side of the hooded means.

In still another embodiment, an electrical box with a removable cover plate is attached to the exterior of one end of the hood. In yet another embodiment, the hood has a downwardly bent lip. In yet another embodiment, the system has six UV light sources and seven target rods in generally parallel orientation. In an alternative embodiment, there are nine UV light sources and eight target rods in generally parallel orientation. In still another embodiment the target rods are of modular construction.

In yet another embodiment, a UV light source is located within an assembly including a reflector tube and a shield, and the assembly is of modular construction. In still another embodiment, the target rods and the assemblies are easy to manufacture, maintain and replace.

In yet another alternative embodiment, the system includes a rigid frame. In still another embodiment, the hood has an adjustable light curtain to at least partially reduce radiation emitted from the UV light sources away from the food. In still another embodiment, the system has a ballast housing and a control box located on the frame, and the frame has wheels. In still another embodiment, the system has an ozone monitor and an alarm adapted to go off at a predetermined ozone level. In still another embodiment, the frame is adaptable to allow the hooded means to be optimally located in relation to the food.

In still another embodiment, the invention is a food sanitation hood, comprising means for subjecting food to sanitizing radiation, means for subjecting food to ozone; and means for subjecting food to hydroxyl radicals, whereby the food is subjected to the radiation, the ozone and the hydroxyl radicals generally simultaneously. In yet another embodiment, the means for subjecting food to sanitizing radiation, the means for subjecting food to ozone and the means for subjecting food to hydroxyl radicals includes one or more sanitizing radiation sources located in an assembly and one or more target rods in optical proximity to the assemblies. In another embodiment, the assemblies and the target rods are modular in construction, as well as easy-to-clean and easy-to-maintain. In still another embodiment, the hood is generally portable. In yet still another embodiment, the hood has a means for attaching the hood to a fixed point on an assembly line. In yet another embodiment, the invention is a method for sanitizing food utilizing a modular, adjustable, portable, easy-to-maintain hood system, comprising the exposing of a food surface simultaneously to UV light, ozone, and hydroxyl radicals.

It is an object of this invention to provide a modular system that makes utilization of the sanitizing combination of radiation, ozone and hydroxyl radicals easy to apply or manufacture economically, or to retrofit into an existing system.

It is also an object of this invention to provide a system designed to be modular, to suit different processes properly, and to provide easy manufacture or replacement of component parts.

It is also an object of this invention to provide a system wherein the radiation, ozone and hydroxyl radicals are optimized for food treatment, efficiently using radiation, ozone and hydroxyl radicals to sanitize processed and/or prepackaged foods.

It is another object of this invention to add moisture to the sanitizing system to improve the efficiency of the formation of hydroxyl radicals in sanitizing food.

It is still another object of this invention to provide a system that is easy to assemble, adjust, transport, clean, maintain and disassemble.

It is yet another object of the invention to provide a system using sanitizing radiation, ozone and hydroxyl radicals that is adjustable on a frame, and that has an adjustable light curtain so high levels of the radiation may be used in a small amount of space and still be safe for operators.

There is no system that takes advantage of the sanitizing and free-moving characteristics of ozone and hydroxyl radicals to improve sanitization beyond what mere radiation can perform.

In accordance with these and other objects which will become apparent hereinafter, the instant invention will now be described with particular reference to the accompanying drawings.

### BRIEF DESCRIPTION OF DRAWINGS

FIG. 1 is a front perspective view of one embodiment of the invention.

FIG. 2 is a partial plan view of the UV light source, shield and target rod as found under the hood portion of the invention.

FIG. 3 is a partial perspective view of the interior of the hood portion of the invention.

FIG. 4 is a rear plan view of one embodiment of the invention, with the light curtain lowered.

FIG. 5 is a partially exploded perspective view of the hood of the invention.

FIG. 6 is a perspective view of an alternative embodiment of the hood portion of the invention.

FIG. 7 is a plan view of an open control panel of the invention.

FIG. 8 is a plan view of an open ballast housing of the invention.

FIG. 9 is a bottom plan view of the preferred embodiment of the invention.

FIG. 10 is a cut away right end plan view of the preferred embodiment of the invention.

FIG. 11 is a left end plan view of the preferred embodiment of the invention.

FIG. 12 is a partially cut away side plan view of the preferred embodiment of the invention.

FIG. 13 is a bottom plan view of an alternative embodiment of the invention.

FIG. 14 is a side plan view of a UV light source of the invention.

FIG. 15A is a side plan view of a reflecting tube of the invention.

FIG. 15B is an end plan view of a reflecting tube of the invention.

FIG. 16 is a side plan view of a shield of the invention.

FIG. 17 is a side plan view of a UV light assembly of the invention.

FIG. 18 is a front plan view of a mounting tab of the invention.

FIG. 19 is a front plan view of a connector tab of the invention.

FIG. 20A is a side plan view of a mounting assembly of the invention.

FIG. 20B is a front plan view of a mounting assembly of the invention.

### DETAILED DESCRIPTION

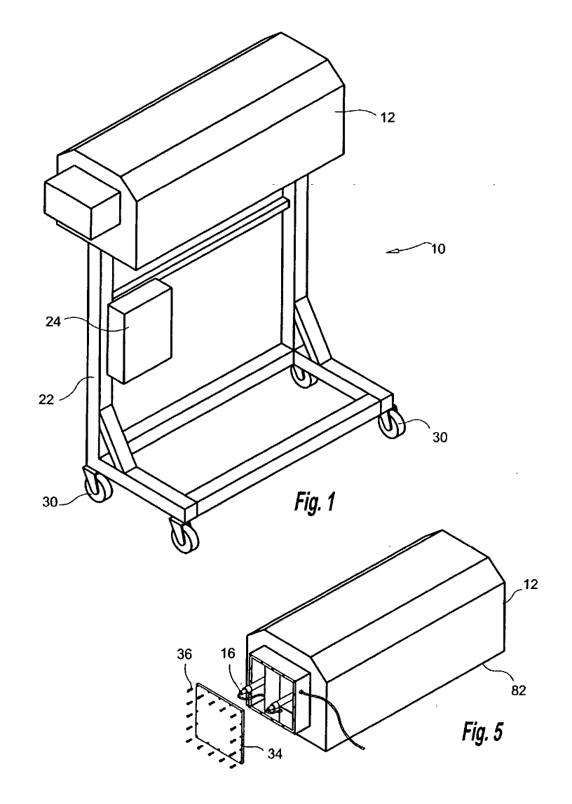

The present invention is a modular, adjustable, portable easy to maintain food sanitation hood system, shown generally in FIG. 1 as 10. Preferably the hood portion 12 is made from a rigid, food safe material, such as stainless steel. It is preferred that food is located under the hood portion 12 or passes below the hood portion 12 along a conveyor belt. The hood is situated so that the surface of the food to be sanitized is facing the hood portion 12.

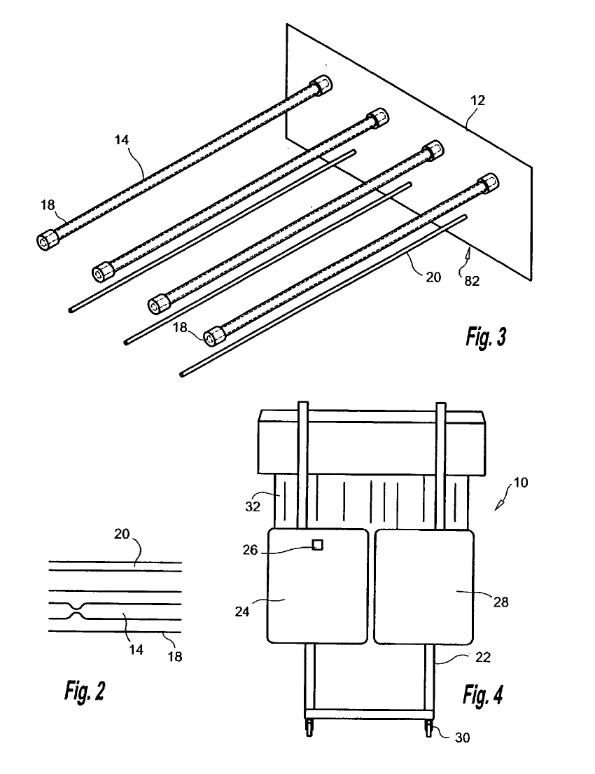

Under the hood portion 12, as shown in FIGS. 2 and 3, is at least one sanitizing radiation source, such as generally cylindrical UV light sources 14. Preferably, as indicated by the neck in the UV light source 14 in FIG. 2, the UV light sources 14 are low vapor mercury lamps that emit UV light of approximately 185 to 254 nm. It is also preferred that the UV light source 14 has its electrical connectors 16 on one end rather than on both ends of the UV lamps 14, as shown in FIG. 5. This configuration facilitates cleaning of the system 10 and replacement of the UV light sources 14 and target rods described below. However, other sanitizing radiation sources such as medium vapor mercury lamps may be used.

Also, around the circumference of the UV light source 14 is preferably a generally cylindrical tubular shield 18, to shield sanitized food passing or lying below the hood portion 12 from any material that may possibly fall onto the food if the UV light source 14 were to break. The shield 18 also protects the UV light source 14 during maintenance and cleaning of the system 10. The shield 18 preferably is made from a fluoro-polymer and is transparent. The UV light sources 14 are recessed from the bottom edge 82 of the hood portion 12 to reduce the emission of UV light from the UV light sources 14 to the area beyond the hood portion 12.

In optical proximity to the UV light sources 14 and under and within the edge 82 of the hood portion 12 is at least one generally cylindrical target rod 20. The target rods 20 are generally parallel to the UV light sources 14. The target rod 20 preferably comprises approximately up to 0-30% titanium dioxide, up to 0-30% silver and up to 0-30% copper, by weight. The UV light itself helps sanitize the food. The high energy UV light also interacts with oxygen to form ozone to also help sanitize the food. In addition, the UV light interacts with moisture and the target rod 20, thus forming hydroxyl radicals to help sanitize the food. The moisture for use with the target rods 20 may exist naturally in the area of the hood portion 12. Alternatively, moisture may be added by a separate mister, either attached to or in the proximity of the hood portion 12.

In one embodiment, shown in FIGS. 1, 4, and 6, the hood portion 12 is attached to the upper section of a rigid frame 22. The hood portion 12 is adjustable vertically and horizontally in relation to the frame 12 to optimize sanitation of the food. Adjustment may be made hydraulically, pneumatically, electronically or mechanically or by other equivalent means. It is preferred that the frame 22 includes an impermeable but easily accessible control box 24 with an on-off switch 26 for the UV lights and a ballast housing 28 for the circuitry for the UV lights. As shown in FIGS. 7 and 8, ballast circuits 38 may be located in both the control box 24 and the ballast housing 28. However, it is preferred that the ballast circuits have separate housing. Also, the control box 24 may include a lamp indicator light 70 to show whether power is being sent to the system 10. It is preferred that the frame 22 and cover panels to the control box 24 and ballast housing 28 are made of an easily cleanable material, such as stainless steel or aluminum. In an alternative embodiment, the frame 22 is located on wheels 30. Preferably, the wheels 30 are able to lock into position so the system 10 is portable yet fixable in a predetermined place.

In addition, as shown in FIG. 4, an adjustable light curtain 32 may be attached to the bottom portion of the hood. When lowered, the light curtain extends below the edge 82 of the hood portion 12. The curtain 32 thereby minimizes any possible exposure of the sanitizing radiation to an operator. The curtain 32 may be made from a flexible sheeting attached to the underside of the hood portion 12 by an adhesive or equivalent means. The sheeting is preferably made from an opaque material. It is also preferred that the curtain 32 extends all the way around the hood portion 12. However, a partial curtain 32 may also be used, and the curtain may alternatively be non-adjustable.

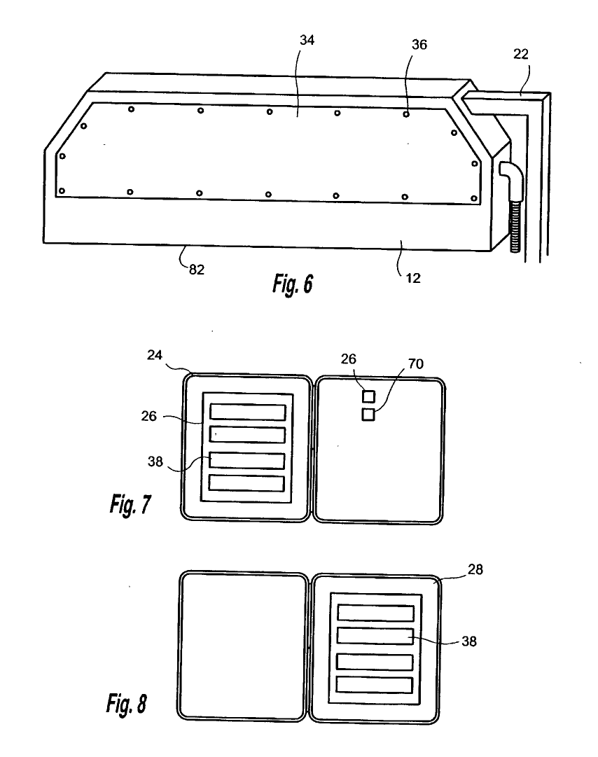

Also, as shown in FIGS. 5 and 6, it may be optionally preferred that the hood portion 12 further includes at least one removable panel 34 fastened by fastening means such as screws 36. The removable panel 34 facilitates cleaning of the system 10 and maintenance and replacement of the UV light sources 14 and target rods 20. The removable panel 34 may be located on the end of the hood portion 12 as shown in FIG. 5, or along the side of the hood portion 12 as shown in FIG. 6, or both.

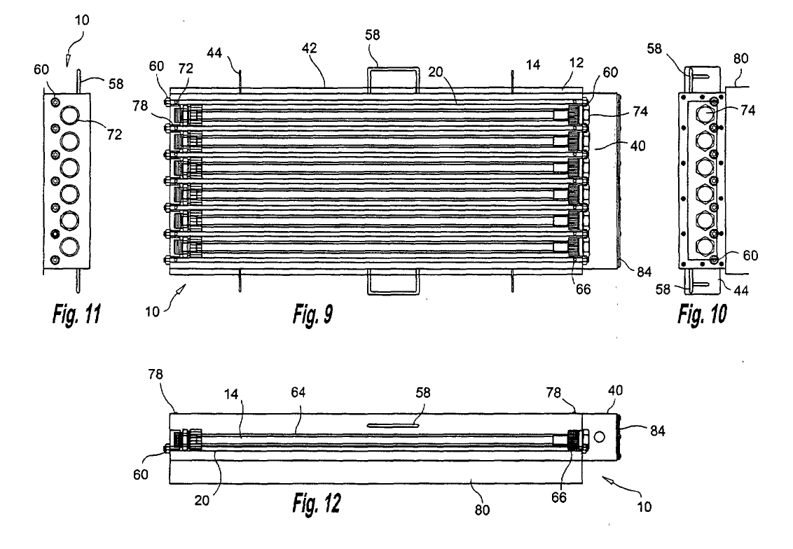

A preferred embodiment of the invention is illustrated in FIGS. 9-12. As shown, the hood portion 12 is generally planar, and made of highly compact, modular components. Furthermore, this embodiment may be installed on an assembly line so the radiation is emitted downward. However, it may also be flipped so that the radiation is emitted upward and a bottom surface of food may be sanitized. For this purpose, the top of the hood portion 12 preferably includes drainage holes 78, in case liquid accumulates onto or into the hood portion 12 during operation. In addition, as shown in FIG. 10, the hood portion 12 also preferably includes a downward bent lip 80 to help contain the sanitizing agents and minimize exposure to users.

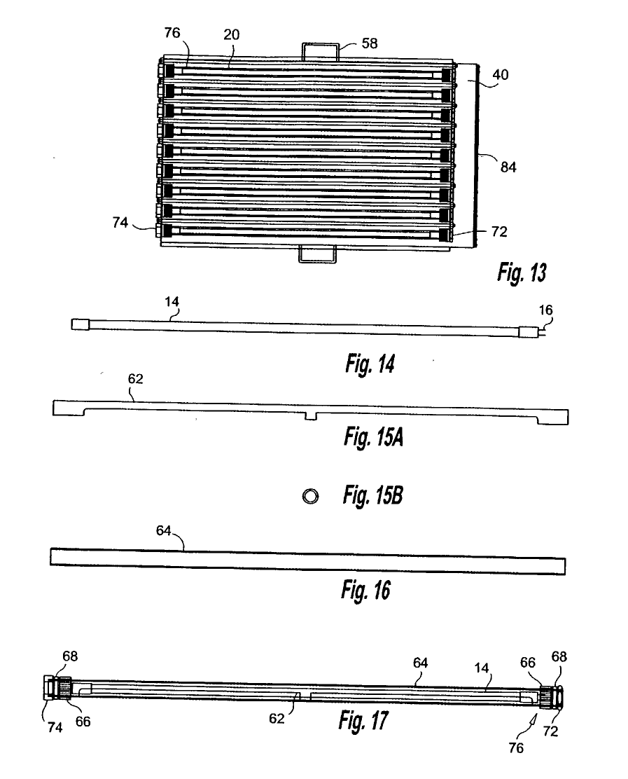

This embodiment comprises at least one UV light source 14 in optical proximity to at least one generally cylindrical target rod 20, both located under and within the circumference of the hood portion 12. The target rod 20 may be attached to a stainless steel rod substrate. The number of target rods 20 and UV light sources 14 is dependent upon the amount of sanitization desired as well as the width of the area to be sanitized. As shown in FIGS. 9-12 and in the alternative embodiment in FIG. 13, the target rods 20 are horizontally offset and evenly interspersed between the UV light sources 14. However, other geometry, such as concave slope, may be preferred for different sanitization processes.

As shown in FIGS. 9-13, target rods 20 are placed within the hood portion 12, and are held in place under the hood portion 12 by endcaps 60 located on the outer surface of the hood portion 12. Thus the target rods 20 are both modular and easily accessible for replacement or maintenance.

A UV light source 14 is preferably in an assembly as illustrated in FIGS. 9 and 12-17. Around the light 58 is a generally cylindrical reflector tube 62, as shown in FIGS. 15A and 15B. The tube 62 has a highly reflective interior surface, so the UV light from the UV light source 14 is used more efficiently onto the target rod 20 and the food being sanitized. Also, it is preferred that the tube 62 be rigid to help in securing the connection of the UV light source 14 to the interior of the hood portion 12. The reflector tube 62 may be positioned so that UV light emitted by the UV light source 14 away from the food is redirected generally toward the target rod 20, the air around the food, and/or the food itself, to optimize the sanitization process. It is also preferred that the reflector tube 62 is of a shape designed so that radiation to the food from the UV light source 14 is not obstructed. In addition, the interior geometry of the reflector tube may be altered to optimize reflection of the UV light onto the target rod 20, the air around the food, or the food itself. The preferred material for the tube 62 is aluminum; however, other metals may be used, or a reflective coating may be placed on the interior surface of a rigid, generally cylindrical piece.

Around the tube 62 is preferably a shield 64, shown in FIG. 16. The shield 64 protects the food from any material that may possibly fall into the food if the UV light source 14 breaks. Preferably, the shield 64 is a transparent, generally flexible cylinder made from fluoro-carbon. However, other equivalent materials may be used.

In the preferred embodiment, as shown in FIGS. 9-13 and 17, on each end of the shield 64 is an endcap fitting 66. Each endcap fitting 66 is generally cylindrical and allows each end of the shield 64 to fit at least partway within it. Around each endcap fitting 66 is a means for forming a compressive seal, such as a gasket 68. On the far end of each endcap fitting 66 is an endcap. A closed endcap 72 is preferred for the end of the UV light source 14 that has no electrical connectors 70 to protect the UV light source 14 and to reduce radiation escaping from the hood portion 12. An open endcap 74 is preferred for the end of the UV light source 14 that has electrical connectors 16.

As shown in FIG. 9, the endcaps 72, 74 do not need to be located on the outside surface of the food portion 12. However, as shown in FIG. 13, to facilitate access to the UV light sources 14, the endcaps 72, 74 may be on the outside surface of the hood portion 12. An open endcap 74 facilitates access to wiring (not shown) of the UV light source 14 to the circuitry in the control box 40 attached to the outer surface of the hood portion 12, as shown in FIGS. 9-13. The control box 40 houses ballast circuits and preferably includes a removable cover plate 84 for easy access to the modular components inside the hood portion 12 as well as the circuitry. In the preferred embodiment, the endcap fittings 66 and the endcaps 72, 74 have complementary threads, so that screwing the endcaps 72, 74 onto the endcap fittings 66 compresses the compressive seal 68. The UV light assembly 76, including endcaps 72, 74, is shown in FIG. 17.

In the preferred embodiment, UV light assemblies 76 and the target rods 14 are fit onto the hood portion 12 as shown in FIGS. 9-12. Compression of the gasket 66 around the endcap fitting 64 helps seal the UV light source 14 into the hood portion 12. The rigid reflector tube 62 helps provide resistance for screwing the endcaps 72, 74 onto the endcap fittings 64, to form the compressive seal. The reflector tube 62 also forms a support for the shield 64 so that the shield 64 does not contact the UV light source 14 during maintenance, thereby protecting the UV light source 14 from breakage.

Attached to the outer surface of the hood portion 12 is preferably a closed electrical box 40 that houses the circuitry and the ballast circuits 38. Also, it is preferred that the box 40 has a removable coverplate 84. Although only one box 40 is shown, the ballast circuits and the circuitry may be located in several attached boxes.

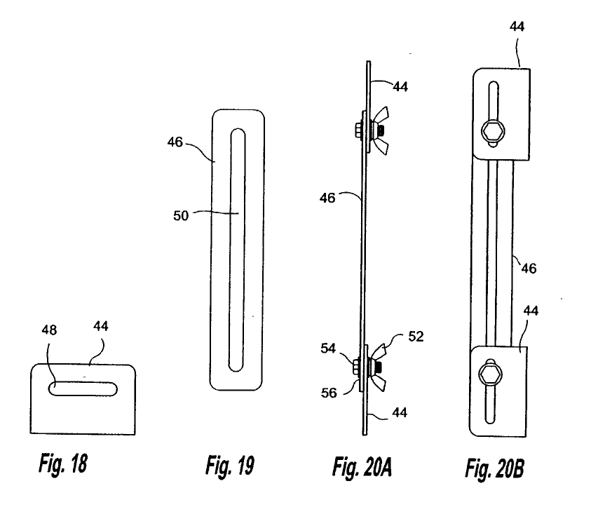

In one alternate embodiment, on each side 42 of the hood portion 12 is at least one generally planar mounting tab 44, as shown in FIGS. 9, 13, and 18, for mounting the hood portion 12 above an area to be sanitized; however, the preferred number of mounting tabs 44 is two on each side. It is also preferred that the mounting tabs 44 each have an inner void 48. The mounting tabs 44 are preferably rigid and made from an easy to maintain and replace material, such as stainless steel or aluminum. It is preferred that the mounting tabs 44 are attached to the outer surface of the hood portion 12 by means such as welding or the equivalent. However, they alternatively may be attached at any point where lifting of the hood may be accomplished. To make mounting of the hood portion 12 easier, it may be preferred that a plurality of mounting tabs 44 are connected by a connector tab 46, as shown in FIGS. 19, 20A, and 20B. As shown, the connector tab 46 has an inner cavity 50 running generally along the length of the connector tab 46. The connector tab 46 is then joined to the mounting tabs 44 on one side of the hood portion 12 by attachment means connecting the void 48 in each mounting tab 44 to the cavity 50 in the connector tab 46 to form a mounting assembly, as shown in FIGS. 20A and 20B. It is preferred that the mounting tabs 44 are adjustably connected to the connector tab 46 by means such as a nut and bolt combination or equivalent means, such as rivets. However, the connector may alternatively be non-adjustable.

As shown in FIGS. 20A and 20B, the mounting tabs 44 are connected to the connector tab 46, preferably by a combination of a wing nut 52 and a bolt 54, where the bolt head is wider that the void 48 or the cavity 50. Washers 56 may be used to facilitate the adjustable and releasable connection. Other equivalent means for mounting the hood portion 12 to sanitize a specific area, such as fixedly mounting the top of the hood portion 12 to a structure, may be performed.

In another embodiment, as shown in FIGS. 9-13, one or more lifting handles 58 may be located on the sides 42 of the hood portion 12. The lifting handle 58 may be permanently or removably attached to the side 42 of the hood portion 12. The lifting handles 58 are preferably made of a rigid, easily cleanable material, such as stainless steel or aluminum, and are preferably welded to the side 42 of the hood portion 12.

Furthermore, the embodiments shown in FIGS. 9-13 may also be used in conjunction with a mister to increase the amount of moisture in the presence of the target rods 20. The mister may be attached to the hood portion 12 or in proximity to it, thereby facilitating hydroxyl radical production.

The instant invention has been shown and described herein in what is considered to be the most practical and preferred embodiment. It is recognized, however, that departures may be made therefrom within the scope of the invention and that obvious modifications will occur to a person skilled in the art.

What is claimed is:

1. A modular, adjustable, portable, easy to maintain food sanitation hood system, comprising:
a hooded means for subjecting food to sanitizers including UV light, ozone and hydroxyl radicals, having:
one or more UV light sources; and
one or more target rods located under the hooded means in optical proximity to the UV light sources.

2. The system of claim 1, wherein the UV radiation-sources emit UV light of approximately 185 to 254 nm.

3. The system of claim 2, wherein the UV radiation sources are at least one of the following: low-vapor mercury and high-vapor mercury UV light sources that emit UV light of approximately 185 to 254 nm.

4. The system of claim 1, wherein the hooded means further comprises drainage holes through a top surface.

5. The system of claim 1, wherein the target rods comprise up to approximately up to 0-30% titanium dioxide, up to 0-30% silver and up to 0-30% copper, by weight.

6. The system of claim 1, further comprising a mister for adding mist in proximity to the target rods for the efficient production of hydroxyl radicals.

7. The system of claim 1, wherein hydroxyl radicals are generated in part from the moisture in the ambient air in the proximity of the target rods.

8. The system of claim 1, further comprising at least one mounting tab located on the outer surface of one side of the hooded means.

9. The system of claim 8, further comprising a connector tab connecting at least two mounting tabs on one side of the hooded means.

10. The system of claim 1, further comprising an electrical box attached to the exterior of one end of the hooded means.

11. The system of claim 10, wherein the electrical box further comprises a removable cover plate.

12. The system of claim 1, wherein the hooded means further comprises a downwardly bent lip.

13. The system of claim 1, further comprising six UV light sources and seven target rods in generally parallel orientation.

14. The system of claim 1, further comprising nine UV light sources and eight target rods in generally parallel orientation.

15. The system of claim 1, wherein the target rods are of modular construction.

16. The system of claim 1, wherein a UV light source is located within an assembly including:
a reflector tube; and
a shield.

17. The system of claim 16, wherein the assembly is of modular construction.

18. The system of claim 17, wherein the target rods and the assemblies are easy to manufacture, maintain and replace.

19. The system of claim 1, further comprising a rigid frame for the hooded means.

20. The system of claim 19, wherein the hooded means includes an adjustable light curtain to at least partially reduce radiation emitted from the UV light sources away from the food.

21. The system of claim 19, further comprising:
a ballast housing; and
a control box located on the frame.

22. The system of claim 19, wherein the frame further comprises wheels.

23. The system of claim 19, further comprising:
an ozone monitor; and
an alarm adapted to go off at a predetermined ozone level.

24. The system of claim 19, wherein the frame is adaptable to allow the hooded means to be optimally located in relation to the food.

25. A food sanitation hood, comprising:
means for subjecting food to sanitizing radiation;
means for subjecting food to ozone; and means for subjecting food to hydroxyl radicals;
whereby the food is subjected to the radiation, the ozone and the hydroxyl radicals generally simultaneously.

26. The hood of claim 25, wherein the means for subjecting food to sanitizing radiation, the means for subjecting food to ozone and the means for subjecting food to hydroxyl radicals includes:
one or more sanitizing radiation sources located in an assembly; and
one or more target rods in optical proximity to the assemblies.

27. The hood of claim 26, wherein the assemblies and the target rods are modular in construction.

28. The hood of claim 27, wherein the assemblies and the target rods are easy-to-clean and easy-to-maintain.

29. The hood of claim 25, wherein the hood is generally portable.

30. The hood of claim 29, further comprising:
means for attaching the hood to a fixed point on an assembly line.

31. A method for sanitizing food utilizing a modular, adjustable, portable, easy-to-maintain hood system, comprising the exposing of a food surface simultaneously to UV light, ozone, and hydroxyl radicals.
